(19) United States
(12) Patent Application Publication
(10) Pub. No.: US 2024/0167019 A1
(43) Pub. Date: May 23, 2024

(54) APTAMERS, CONTAINERS AND METHODS FOR CELL TRANSDUCTION

(71) Applicant: SAINT-GOBAIN PERFORMANCE PLASTICS CORPORATION, Solon, OH (US)

(72) Inventors: Katie Campbell, Northborough, MA (US); Natalie Fekete, Worcester, MA (US); Jonathon Wheatley, Traverse City, MI (US); Jessica Rodriguez, Jacobus, PA (US); Maranda Gibb, Jacobus, PA (US); Albert M. Liao, Jacobus, PA (US); Thomas Caltagirone, Jacobus, PA (US)

(21) Appl. No.: 18/514,893

(22) Filed: Nov. 20, 2023

Related U.S. Application Data
(60) Provisional application No. 63/384,354, filed on Nov. 18, 2022.

(51) Int. Cl. C12N 15/10; C12M 1/00; C12M 1/12; C12N 15/115; C12N 15/86

(52) U.S. Cl. CPC C12N 15/1082; C12M 23/14; C12M 25/14; C12N 15/1048; C12N 15/115; C12N 15/86; C12N 2310/16; C12N 2740/15043

(57) ABSTRACT

This disclosure relates generally to nucleic acid aptamers especially useful for cell transduction, as well as to containers (such as bags) having surfaces comprising one or such aptamers, and to transductions methods using such aptamers and containers. One embodiment of the disclosure provides A DNA aptamer comprising a plurality of nucleotides, the DNA aptamer having at least 80% sequence identity with the sequence of SEQ ID NO: 1 (AAACTGCAGCGATTCATTAGTACGGCCTTT).

### CROSS-REFERENCE TO RELATED APPLICATIONS

This application claims the benefit of priority of U.S. Provisional Patent Application No. 63/384,354, filed Nov. 18, 2022, which is hereby incorporated herein by reference in its entirety.

### INCORPORATION BY REFERENCE OF SEQUENCE LISTING PROVIDED ELECTRONICALLY

This application contains a Sequence Listing submitted as an electronic text file named “22-1286-US_SequenceListing_ST26.xml,” having a size of 2894 bytes, and created on Nov. 20, 2023. The information contained in this electronic file is hereby incorporated by reference in its entirety.

### BACKGROUND OF THE DISCLOSURE

### Field of the Disclosure

This disclosure relates generally to nucleic acid aptamers especially useful for cell transduction, as well as to containers (such as bags) having surfaces comprising one or such aptamers, and to transductions methods using such aptamers and containers.

### Technical Background

Transduction, the process by which foreign DNA is introduced into a target cell, e.g., by a viral vector, is important in a number of applications. For example, CAR T-cell therapy is a cancer treatment in which a gene encoding a chimeric antigen receptor (CAR) is introduced to T cells collected from a patient by transduction. The modified cells, after expansion and re-introduction to the patient, can bind to and kill cancer cells. Additionally, viral transduction is regularly used in basic genetic research.

However, gene transfer from a viral vector can be inefficient for certain cell types, such as hematopoietic cells and other suspension cells. Conventionally, transduction efficiency for such systems can be improved by use of an enhancer such as polybrene, protamine sulfate, or retronectin. Polybrene and protamine sulfate improve transduction efficiency by modifying the surface properties of target cells. However, these types of enhancers can negatively impact cell viability. Alternatively, retronectin—a polypeptide that includes a heparin-binding domain having a binding affinity for viral particles and two cell-binding domains having a binding affinity for VLA-4 and VLA-5 surface receptors—can improve transduction efficiency by facilitating co-localization of viral vectors and target cells.

Conventionally, transduction enhancers such as retronectin must be manually coated onto a container before a transduction process. Because the coated containers require special refrigeration and have a short shelf life, the relatively costly, time-consuming coating process typically must be performed by the user conducting the transduction process, shortly before the process.

Accordingly, there remains a need for simple, cost-effective, and/or time-effective system for cell transduction.

### SUMMARY OF THE DISCLOSURE

In one aspect, the disclosure provides a DNA aptamer having at least 80% sequence identity with the sequence of SEQ ID NO: 1 (i.e., AAACTGCAGCGATTCATTAGTACGGCCTTT).

In another aspect, the disclosure provides a DNA aptamer having at least 80% sequence identity with the sequence of SEQ ID NO: 2 (i.e., CGAGGCTCTCGGGACGACAAACTGCAGCGATTCATTAGTACGGCCTTTGTCGTCCCGC CTTTAGGATTTACAG).

In one aspect, the disclosure provides a surface for cell transduction, having a DNA aptamer as described herein disposed thereon, e.g., by covalent bonding.

In another aspect, the disclosure provides a container (e.g., in the form of a bag) having an outer surface and an inner surface, the inner surface comprising
a polymer, e.g., a fluoropolymer;
attached to the polymer, a plurality of functional groups; and
attached to each of at least a portion of the functional groups, a DNA aptamer as described herein.

In another aspect, the disclosure provides a transduction method, comprising contacting a viral vector and a target cell with a surface in a container as described herein.

Other aspects of the disclosure will be apparent to the person of ordinary skill in the art in view of the disclosure herein.

### BRIEF DESCRIPTION OF THE DRAWINGS

FIG. 1 is a schematic top-down (top) and cross-sectional (bottom) view of a bag according to one embodiment of the disclosure.

FIG. 2 is a schematic top-down view of bags according to various embodiments of the disclosure.

FIG. 3. is a diagram illustrating a process of melting-off aptamer selection.

FIG. 4 is a graph showing enrichment of aptamer.

FIG. 5 is a diagram illustrating a process of parallel assessment.

FIG. 6 is a diagram illustrating a process of bioinformatics & candidate selection.

FIG. 7 is a graph of distribution of family frequencies from the P191(+) G11(P) library that do not Appear in G11(C).

FIG. 8 is a set of graphs of SPR-based assessment of SGO-P06701 against target and counter-target

### DETAILED DESCRIPTION

The present inventors have noted that cell transduction can be performed on surfaces that bear aptamers have binding affinity for the cell receptor VLA-4, which is found at the surface of a variety of cell types, such as stem cells, progenitor cells, T and B cells, monocytes, natural killer cells, and eosinophils.

The present inventors have now identified a set of DNA aptamers that can have high affinity for binding the VLA-4 surface receptor of a cell. Accordingly, one aspect of the disclosure is a DNA aptamer having at least 80% sequence identity with the sequence of SEQ ID NO: 1 (i.e., AAACTGCAGCGATTCATTAGTACGGCCTTT). In another aspect, the disclosure provides a DNA aptamer having at least 80% sequence identity with the sequence of SEQ ID NO: 2 (i.e., CGAGGCTCTCGGGACGACAAACTGCAGCGATTCATTAGTACGGCCTTTGTCGTCCCGC CTTTAGGATTTACAG). Without intending to be bound by theory, the inventors believe that it is the sequence of SEQ ID NO: 1 that drives the selectivity of binding to VLA-4. As described below in the Examples, various test sequences were positioned between PCR primer annealing regions, in sequences of structure (5′-CGA GGC TCT CGG GAC GAC-[sequence]-GTC GTC CCG CCT TTA GGA TTT ACA G-3′). Accordingly it is believed that the sequence between the PCR primer annealing regions is of primary importance. The sequence of SEQ ID NO: 1 was shown to have high selectivity when in a molecule of the structure above (i.e., which has the sequence of SEQ ID NO: 2). Other sequences disposed between these PCR primer annealing regions provided far less selective binding.

The term “DNA aptamer” (sometimes shortened to “aptamer”) as used herein refers to polydeoxyribonucleotides (i.e., having any number of nucleotides greater than 1) that can bind (e.g. with high affinity and specificity) to a target molecule, typically a protein, peptide, or small molecule. Aptamers typically have defined secondary or tertiary structure owing to their propensity to form complementary base pairs and, thus, are often able to fold into diverse and intricate molecular structures. The three-dimensional structures are essential for aptamer binding affinity and specificity, and specific three-dimensional interactions drives the formation of aptamer-target complexes. Aptamers are typically selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510). However, particular DNA aptamers of the disclosure herein can be made by modification of the sequence of SEQ ID NOS: 1 and 2 and using DNA synthesis techniques familiar to the person of ordinary skill in the art. The Example section provided below describes the development of the aptamer of SEQ ID NOS: 1 and 2.

As used herein, a nucleotide that has a particular % sequence identity to a particular SEQ ID NO: has that % of the nucleotides of the sequence of the SEQ ID NO. in the same relative positions with respect to one another. Such a nucleotide may have more or less nucleotides than the number of nucleotides (i.e., 30 or 73) listed in the SEQ ID NO., as long as it meets the particular sequence identity described.

In various embodiments, the DNA aptamer has at least 80% (i.e., at least 24/30) sequence identity with the sequence of SEQ ID NO: 1. In various embodiments, the DNA aptamer has at least 86.6% (i.e., at least 26/30) sequence identity with the sequence of SEQ ID NO: 1. In various embodiments, the DNA aptamer has at least 90% (i.e., at least 27/30) sequence identity with the sequence of SEQ ID NO: 1. In various embodiments, the DNA aptamer has at least 93.3% (i.e., at least 28/30) sequence identity with the sequence of SEQ ID NO: 1. In various embodiments, the DNA aptamer has at least 96.6% (i.e., at least 29/30) sequence identity with the sequence of SEQ ID NO: 1. In various embodiments, a DNA aptamer comprises the sequence of SEQ ID NO: 1, i.e., it has 100% (30/30) sequence identity with the sequence of SEQ ID NO: 1. In some embodiments, additional nucleotides can be present over and above those listed in SEQ ID NO: 1. However, in other such cases, nucleotides of SEQ ID NO: 1 are the only nucleotides of the DNA aptamer.

Similarly, as used herein, a nucleotide that has given % sequence identity to SEQ ID NO: 2 has at least 80% (i.e., at least 58/73) of the nucleotides of SEQ ID NO: 2 in the same relative positions with respect to one another. Such a nucleotide may have more or less nucleotides than the 73 nucleotides listed in SEQ ID NO: 2, as long as it meets the particular sequence identity described. In various embodiments, the DNA aptamer has at least 85% (i.e., at least 62/73) sequence identity with the sequence of SEQ ID NO: 2. In various embodiments, the DNA aptamer has at least 90% (i.e., at least 66/73) sequence identity with the sequence of SEQ ID NO: 2. In various embodiments, the DNA aptamer has at least 91.7% (i.e., at least 67/73) sequence identity with the sequence of SEQ ID NO: 2. In various embodiments, the DNA aptamer has at least 93.1% (i.e., at least 68/73) sequence identity with the sequence of SEQ ID NO: 1. In various embodiments, the DNA aptamer has at least 94.5% (i.e., at least 69/73) sequence identity with the sequence of SEQ ID NO: 2. In various embodiments, the DNA aptamer has at least 95.8% (i.e., at least 70/73) sequence identity with the sequence of SEQ ID NO: 2. In various embodiments, the DNA aptamer has at least 97.2% (i.e., at least 71/73) sequence identity with the sequence of SEQ ID NO: 2. In various embodiments, the DNA aptamer has at least 98.6% (i.e., at least 72/73) sequence identity with the sequence of SEQ ID NO: 2. In various embodiments, a DNA aptamer comprises the sequence of SEQ ID NO: 2, i.e., it has 100% (73/73) sequence identity with the sequence of SEQ ID NO: 2. In some embodiments, additional nucleotides can be present over and above those listed in. However, in other such cases, nucleotides of SEQ ID NO: 2 are the only nucleotides of the DNA aptamer.

It can be desirable for relatively large stretches of the sequence of SEQ ID NO: 1 or SEQ ID NO: 2 to be present in a DNA aptamer as otherwise described herein. For example, in various embodiments, a DNA aptamer as otherwise described herein has at least one span of 10 contiguous nucleotides in common with the sequence of SEQ ID NO: 1, e.g., one such span, or two such spans, three such spans. In various embodiments, a DNA aptamer as otherwise described herein has at least one span of 15 contiguous nucleotides in common with the sequence of SEQ ID NO: 1, e.g., one such span or two such spans. In various embodiments, a DNA aptamer as otherwise described herein has a span of 20 contiguous nucleotides in common with the sequence of SEQ ID NO: 1. In various embodiments, a DNA aptamer as otherwise described herein has a span of 25 contiguous nucleotides in common with the sequence of SEQ ID NO: 1.

Similarly, in various embodiments, a DNA aptamer as otherwise described herein has at least one span of 10 contiguous nucleotides in common with the sequence of SEQ ID NO: 2, e.g., one such span, or two such spans, or three such spans, or four such spans, or five such spans, or six such spans. In various embodiments, a DNA aptamer as otherwise described herein has at least one span of 20 contiguous nucleotides in common with the sequence of SEQ ID NO: 2, e.g., one such span, or two such spans, or three such spans. In various embodiments, a DNA aptamer as otherwise described herein has at least one span of 30 contiguous nucleotides in common with the sequence of SEQ ID NO: 2, e.g., one such span, or two such spans. In various embodiments, a DNA aptamer as otherwise described herein has a span of 40 contiguous nucleotides in common with the sequence of SEQ ID NO: 2. In various embodiments, a DNA aptamer as otherwise described herein has a span of 50 contiguous nucleotides in common with the sequence of SEQ ID NO: 2. In various embodiments, a DNA aptamer as otherwise described herein has a span of 60 contiguous nucleotides in common with the sequence of SEQ ID NO: 2. In various embodiments, a DNA aptamer as otherwise described herein has a span of 70 contiguous nucleotides in common with the sequence of SEQ ID NO: 2.

The DNA aptamer as otherwise described herein can have a variety of lengths. For example, in various embodiments, a DNA aptamer having at least 80% sequence identity with the sequence of SEQ ID NO: 1 as otherwise described herein has at least 24 nucleotides, e.g., at least 26 nucleotides, or at least 27 nucleotides, or at least 28 nucleotides, or at least 29 nucleotides, or at least 30 nucleotides. In various embodiments, a DNA aptamer having at least 80% sequence identity with the sequence of SEQ ID NO: 1 as otherwise described herein has no more than 200 nucleotides, e.g., no more than 100 nucleotides, or no more than 75 nucleotides, or no more than 50 nucleotides, or no more than 40 nucleotides, or no more than 35 nucleotides, or no more than 30 nucleotides. In various embodiments, a DNA aptamer having at least 80% sequence identity with the sequence of SEQ ID NO: 1 as otherwise described herein has in the range of 24-200 nucleotides, e.g., 26-200, or 27-200, or 28-200, or 29-200, or 30-200, or 24-150, or 26-150, or 27-100, or 28-100, or 29-100, or 30-100, or 24-75, or 26-75, or 27-75, or 28-75, or 29-75, or 30-75, or 24-50, or 26-50, or 27-50, or 28-50, or 29-50, or 30-50, or 24-40, or 26-40, or 27-40, or 28-40, or 29-40, or 30-40 nucleotides. For example, in some embodiments, a DNA aptamer having at least 80% sequence identity with the sequence of SEQ ID NO: 1 as otherwise described herein has in the range of 24-36 nucleotides, e.g., 26-36, or 28-36, or 30-36, or 24-34, or 26-34, or 28-34, or 30-34, or 24-32, or 26-32, or 28-32, or 30-32, or 24-30, or 26-30, or 28-30, or 30 nucleotides.

Similarly, in various embodiments, a DNA aptamer having at least 80% sequence identity with the sequence of SEQ ID NO: 2 as otherwise described herein has at least 60 nucleotides, e.g., at least 65 nucleotides, or at least 70 nucleotides, or at least 71 nucleotides, or at least 72 nucleotides, or at least 73 nucleotides. In various embodiments, a DNA aptamer having at least 80% sequence identity with the sequence of SEQ ID NO: 2 as otherwise described herein has no more than 200 nucleotides, e.g., no more than 150 nucleotides, or no more than 100 nucleotides, or no more than 80 nucleotides, or no more than 75 nucleotides, or no more than 74 nucleotides, or no more than 73 nucleotides. In various embodiments, a DNA aptamer having at least 80% sequence identity with the sequence of SEQ ID NO: 2 as otherwise described herein has in the range of 60-200 nucleotides, e.g., 65-200, or 70-200, or 71-200, or 72-200, or 73-200, or 60-150, or 70-150, or 71-150, or 72-150, or 73-150, or 60-100, or 70-100, or 71-100, or 72-100, or 73-100, or 60-80, or 70-80, or 71-80, or 72-80, or 73-80, or 60-75, or 65-75, or 70-75, or 60-74, or 65-74, or 70-74, or 60-73, or 65-73 or 70-73 nucleotides. For example, in some embodiments, a DNA aptamer having at least 80% sequence identity with the sequence of SEQ ID NO: 2 as otherwise described herein has in the range of 71-75 nucleotides, e.g., 72-75, or 73-75, or 71-74, or 72-74, or 73-74, or 71-73, or 72-73, or 73 nucleotides.

A DNA aptamer as described herein can include only a single instance of the sequence having a desired % identity with a particular sequence (i.e., of SEQ ID NO: 1 or SEQ ID NO: 2) described here. However, in other embodiments, a DNA aptamer has multiple repeats of such a sequence, e.g., at least 2, at least 5, at least 10, or at least 25, or at least 50, or at least 75, or at least 100, or at least 200, or at least 300, or at least 400, or at least 500, or at least 750, or at least 1000 repeats of the sequence. Polynucleotides comprising multiple repeats of given aptamer sequences can be made via rolling circle amplification (RCA) of a template corresponding to the desired aptamer sequence.

In various embodiments as otherwise described herein, the DNA aptamer has a binding affinity for VLA-4 of at least 0.1 nM. For example, in certain such embodiments, the DNA aptamer has a binding affinity for the VLA-4 of at least 0.25 nM, or at least 0.5 nM, 0.75 nM, or at least 1 nM, or at least 2.5 nM, or at least 5 nM, or at least 10 nM, or at least 25 nM, or at least 50 nM, or at least 75 nM, or at least 100 nM. As used herein, binding affinity is determined as described in the Examples below. The person of ordinary skill in the art can select a binding affinity that provides for a desirable degree of binding of VLA-4, to allow, e.g., a desirable degree of binding of a cell to be transduced to a surface functionalized with the aptamer.

As noted above, in various embodiments a DNA aptamer of the disclosure is covalently bound to a surface material such as a fluoropolymer. Accordingly, another embodiment of the disclosure is a DNA aptamer as otherwise described herein that also includes reactive group-terminated linker covalently bound to the plurality of nucleotides. The reactive group of the linker can be used to covalently attach the aptamer to a surface.

As the person of ordinary skill in the art will appreciate, a variety of reactive groups can be used, depending especially on the functional groups available for binding on the surface to which the aptamer is to be attached. In various desirable embodiments, the reactive group is a primary amine; the present inventors have determined that reductive amination of aldehydes and ketones, as well as amide coupling using agents such as N-hydroxysuccinimide can be especially desirable chemistries to attach an aptamer to a surface. But the person of ordinary skill in the art will appreciate that a variety of other reactive groups are known for use in covalently bonding nucleotides to other species, including, for example,
(a) carboxyl groups and various derivatives thereof including, but not limited to, N-hydroxysuccinimide esters, N-hydroxybenzotriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.;
(c) haloalkyl groups in which the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups that are capable of participating in Diels-Alder reactions such as, for example, maleimido groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold;
(h) alkenes, which can undergo, for example, cycloadditions, acylation, Michael addition, etc.;
(j) epoxides, which can react with, for example, amines and hydroxyl compounds;
(j) phosphoramidites and other standard functional groups useful in nucleic acid synthesis; and
(l) reactive silanes, which can bond to silicon oxide surfaces like those of quartz and various glasses.

But the person of ordinary skill in the art will appreciate that this is not an exhaustive list.

Similarly, a variety of linkers can be used. As the person of ordinary skill in the art will appreciate, a variety of linking groups can be selected to provide a desired spacing from a surface and to be compatible with the chemistries used to covalently bond to the surface and to covalently bond to the nucleotides of the aptamer. For example, in some embodiments, the linker is a difunctional hydrocarbon or a difunctional polyether. But many, many other choices are possible.

The chemistry used to attach the linker to the nucleotides of the DNA aptamer is similarly not particularly limited. For example, the linker can be connected through a phosphodiester bond to the nucleotides, e.g., to a 5′ end of the nucleotides or a 3′ end of the nucleotides. But other linking chemistries are possible, e.g., by acylation of OH groups of the sugar groups of the deoxyriboses of the aptamer backbone.

Overall, in various embodiments, the linker separates the reactive group from an atom of the nucleotides of the aptamer by no more than 200 bonds, e.g., no more than 150 bonds, or no more than 100 bonds, or no more than 50 bonds, or no more than 25 bonds. In various embodiments, the linker separates the reactive group from an atom of the nucleotides of the aptamer by at least 4 bonds, e.g., at least 6 bonds, or at least 8 bonds, or at least 10 bonds. In various embodiments, the linker separates the reactive group from an atom of the nucleotides of the aptamer by in the range of 4-200 bonds, e.g., 6-200, or 8-200, or 10-200, or 4-150, or 6-150, or 8-150, or 10-150, or 4-100, or 6-100, or 8-100, or 10-100, or 4-50, or 6-50, or 8-50, or 10-50, or 4-25, or 6-25, or 8-25, or 10-25 bonds.

As an example, a variety of primary amine-bearing phosphoramidite linkers are available from ThermoFisher. Reagents such as 6-(4-Monomethoxytritylamino)-hexyl-[(2-cyanoethyl)-(N, N-diisopropyl)]-phosphoramidite; 6-(N-Trifluoroacetylamino)hexyl-[(2-cyanoethyl)-(N,N-diisopropyl)]-phosphoramidite; and 2-[2-(4,4-Dimethoxytrityloxy)ethylsulfonyl]ethyl-[(2-cyanoethyl)-(N,N-diisopropyl)]-phosphoramidite can be used to provide primary amine-terminated linkers bound through phosphodiester bonds to the nucleotides. As the person of ordinary skill in the art will appreciate, these can be used directly in the DNA synthesis process, or can be added at a later time.

In other embodiments, non-covalent binding can be used to attach the DNA aptamer to the surface, for example, using non-covalent associations such as (strep)avidin/biotin.

Another aspect of the disclosure is a functionalized surface that comprises a surface material; and covalently attached to the surface material, a DNA aptamer as described herein. Advantageously, as the DNA aptamers described herein can have high binding affinity for VLA-4, the functionalized surfaces described herein can bind and localize VLA-4 at the surface, where it can be used for transduction.

As the person of ordinary skill in the art will appreciate, a variety of surface materials can be used. In various embodiments, the surface material is a fluoropolymer. A variety of fluoropolymers can be used at the inner surface of the containers as described herein. In various embodiments as otherwise described herein, the inner surface of the container comprises a fluoropolymer selected from polytetrafluoroethylene (PTFE), perfluoroalkoxy (PFA), ethylene tetrafluoroethylene (ETFE), polyvinylidene fluoride (PVDF), polychlorotrifluoroethylene (PCTFE), ethylene chlorotrifluoroethylene (ECTFE), fluorinated ethylene propylene (FEP), ethylene fluorinated ethylene propylene (EFEP), perfluoropolyether (PFPE), modified polytetrafluoroethylene (TFM), polyvinyl fluoride (PVF), or any mixture thereof. For example, in various embodiments as otherwise described herein, the inner surface of the container comprises fluorinated ethylene propylene polymer.

In various embodiments as otherwise described herein, a fluoropolymer material used as a surface material has a thickness of at least 0.0003 inches, at least 0.0004 inches, at least 0.0005 inches, at least 0.0006 inches, at least 0.001 inches, or at least 0.10 inches. For example, in certain such embodiments, a fluoropolymer material used as a surface material has a thickness within the range of 0.0003 inches to 0.2 inches, or 0.0003 inches to 0.1 inches, or 0.0005 inches to 0.08 inches, or 0.001 inches to 0.07 inches, or 0.001 inches to 0.05 inches, or 0.001 inches to 0.03 inches, or 0.001 inches to 0.018 inches, or 0.001 inches to 0.016 inches, or 0.001 inches to 0.014 inches, or 0.001 inches to 0.012 inches.

In various embodiments as otherwise described herein, a fluoropolymer material used as a surface material is a layer of a multilayer material, with a layer of another polymeric material (fluoropolymeric or otherwise) opposite the functionalized surface. In various embodiments as otherwise described herein, the material opposite the functionalized surface of the container has a thickness of at least 0.0005 inches, or at least 0.001 inches, or at least 0.005 inches, or at least 0.0075 inches, or at least 0.01 inches, or at least 0.02 inches, or at least 0.03 inches, or at least 0.04 inches, or at least 0.05 inches, or at least 0.06 inches, or at least 0.07 inches, or at least 0.08 inches, or at least 0.09 inches, or at least 0.1 inches, or at least 0.11 inches. For example, in certain such embodiments, the material opposite the functionalized surface has a thickness within the range of 0.0005 inches to 0.2 inches, or 0.005 inches to 0.18 inches, or 0.01 inches to 0.16 inches, or 0.01 inches to 0.14 inches, or 0.01 inches to 0.12 inches, or 0.06 inches to 0.13 inches, or 0.09 inches to 0.126 inches.

In various embodiments as otherwise described herein the material opposite the functionalized surface is a material other than a fluoropolymer. For example, in certain such embodiments, the material opposite the functionalized surface is a thermoplastic polymer, a thermoplastic elastomer, a silicone, a rubber, or any combination thereof.

A variety of other materials can be provided as a surface material. For example, silicate surfaces like glass or silica can be used; such materials can be functionalized with organosilanes to provide functional groups (e.g., epoxides, carboxylates, amines) which can be reacted with reactive groups of a linker-containing aptamer. Other polymer materials can also be used, e.g., polymers based at least in part on one or more of acrylic acid, vinyl alcohol, ethyleneimine can provide functional groups that can be reacted with reactive groups of a linker-containing aptamer.

While fluoropolymers are typically inert to reaction, the present inventors have noted that they can be functionalized to allow for bonding of aptamers in a number of ways. For example, a plurality of functional groups can be formed on the fluoropolymer surface, followed by reaction of those functional groups with the reactive group of the aptamer. For example, the fluoropolymer of the inner surface may be functionalized with carboxy groups, hydroxyl groups, aldehyde groups, carbonyl groups, amine groups, imine groups, amide groups, ester groups, anhydride groups, thiol groups, disulfides groups, phenol groups, guanidine groups, thioether groups, indole groups, imidazole groups, aminoethyl amide groups, alkyne groups, alkene groups, aziridine groups, epoxy groups, isonitrile groups, isocyanide groups, tetrazine groups, diazonium surface groups, alkyne groups, alkene groups, aziridine groups, epoxy groups, isonitrile groups, isocyanide groups, tetrazine groups, alkyl groups, aminoethyl amide groups, ester groups, or any mixture thereof.

For example, in certain such embodiments, the functional groups include aldehyde groups. Such groups can be reacted with a primary amine to form an imine, which in turn can be reduced (e.g., with borohydride) to form an amine binding the aptamer to the surface.

In various embodiments, the functional groups include carboxylate groups. These can be coupled to a primary amine, e.g., using standard peptide coupling reactions (for example using N-hydroxysuccinimide) to form amide bonds binding the aptamer to the surface.

In various embodiments as otherwise described herein, the functional groups include nitrogen-containing groups. For example, in certain such embodiments, the inner surface of the container comprises a plurality of amine groups, which can be bound to a carboxylate reactive group (e.g., as described above) to form amide bonds binding the aptamer to the surface.

But as described above, the person of ordinary skill in the art will appreciate that a variety of other chemistries is possible.

The person of ordinary skill in the art will appreciate that functional groups can be provided at a fluoropolymer surface in many ways. In various embodiments as otherwise described herein, the functional groups at the inner surface of the container are the product of etching of the fluoropolymer. For example, in certain such embodiments, the etching comprises chemical etching, physical-mechanical etching, or plasma etching. For example, in various embodiments, the functional groups comprising the inner surface are the product of chemical etching of the fluoropolymer. In certain such embodiments, the chemical etching comprises etching with sodium ammonia or sodium naphthalene. In another example, in various embodiments, the functional groups comprising the inner surface are the product of physical-mechanical etching. In certain such embodiments, the physical-mechanical etching comprises sandblasting or air abrasion with silica. In another example, the functional groups comprising the inner surface are the product of plasma etching. In certain such embodiments, the plasma etching comprises etching with reactive plasmas such as hydrogen, oxygen, acetylene, methane, and mixtures thereof with nitrogen, argon, and helium.

In various embodiments as otherwise described herein, the functional groups at the inner surface of the container are the product of activation of the fluoropolymer in the presence of a reactive species. For example, in certain such embodiments, the activation is plasma activation. In various embodiments, plasma activation includes formation of reactive species on the fluoropolymer by treatment with gases such as, for example, argon, hydrogen, nitrogen, carbon dioxide, oxygen and mixtures thereof. In various embodiments, plasma activation generates radicals and/or peroxides on a fluoropolymer. Plasma activation can, in various embodiments, be performed at a pressure within the range of 0.1 Torr to 0.6 Torr, or within the range of 700 Torr to 760 Torr. In another example, in certain such embodiments, the activation is corona activation. In various embodiments, corona activation includes activation of the fluoropolymer under gases such as, for example, argon, nitrogen, hydrogen, and mixtures thereof to form active sites on the fluoropolymer (e.g., susceptible to a reactive species or subsequent chemical treatment). In various embodiments, the activation (e.g., plasma activation or corona activation) includes a reactive hydrocarbon vapor such as, for example, ketones, alcohols, p-chlorostyrene, acrylonitrile, propylene diamine, anhydrous ammonia, styrene sulfonic acid, carbon tetrachloride, tetraethylene pentamine, cyclohexyl amine, tetra isopropyl titanate, decyl amine, tetrahydrofuran, diethyl triamine, tertiary butyl amine, ethylene diamine, toluene-2,4-diisocyanate, glycidyl methacrylate, triethylene tetramine, hexane, triethyl amine, methyl alcohol, vinyl acetate, methylisopropyl amine, vinyl butyl ether, methyl methacrylate, 2-vinyl pyrrolidone, methylvinylketone, xylene, or mixtures thereof. In various embodiments as otherwise described herein, activation (e.g., plasma activation) including a polymerizable hydrocarbon vapor selected from, for example, butylene, ethylene, glutaraldehyde, etc., provides a polymer (i.e., comprising a functional group as otherwise described herein) coated onto the fluoropolymer. The person of ordinary skill in the art will appreciate that, in various embodiments, plasma activation including a polymerizable hydrocarbon vapor (i.e., plasma polymerization) can provide a relatively disorganized, highly cross-linked polymer coating.

In various embodiments as otherwise described herein, the functional groups at the inner surface of the container are the product of chemically treating an activated fluoropolymer. For example, in certain such embodiments, the activated fluoropolymer is the product of plasma activation or corona activation of the fluoropolymer. In certain such embodiments, the chemical treatment is a chemical reaction such as, for example, grafting polymerization, coupling, click chemistry, condensation, or addition. In various embodiments, the chemical treatment is grafting polymerization in solution, comprising polymerizing vinyl monomers via radical polymerization (e.g., initiated by radicals generated through plasma activation of the fluoropolymer). In certain such embodiments, the vinyl monomers are selected from, for example, acrylic acid, (meth)acrylates, (meth)alkylacrylates, styrenes, dienes, alpha-olefins, halogenated alkenes, (meth)acrylonitriles, acrylamides, N-vinyl carbazoles, N-vinyl pyrrolidones, and maleic anhydride. For example, radical polymerization of acrylic acid monomers on the fluoropolymer can, in various embodiments, provide a dense surface of carboxyl groups. In various embodiments, such polymerized products can be relatively organized (e.g., as compared to plasma-polymerized products).

In various embodiments as otherwise described herein, the functional groups at the inner surface of the container are the product of coating an activated fluoropolymer. For example, in certain such embodiments, the activated fluoropolymer is the product of plasma activation or corona activation of the fluoropolymer. In certain such embodiments, the coating is wet coating, powder coating, or chemical vapor deposition. In various embodiments, the coating is plasma-enhanced chemical vapor deposition or initiated chemical vapor deposition.

Plasma polymerization of an alkyl aldehyde such as propionaldehyde is an especially desirable way to provide an aldehyde-bearing fluoropolymer surface.

As described above, the DNA aptamer can be covalently bound to the surface material. For example, in various embodiments as otherwise described herein, the DNA aptamer is covalently bound to the surface material through reaction of a reactive group with a functional group of the surface material. In various embodiments, the reaction of the reactive group with the functional group of the surface material forms a phosphodiester, an amide, or an amine covalently bonding the DNA aptamer to the surface material (e.g., through a linking group as described above).

As noted above, a functionalized surface of the disclosure includes a DNA aptamer of the disclosure bound to a surface material. The present inventors have determined that useful surfaces for cell transduction can also include a second DNA aptamer having a binding affinity for a viral vector bound to the surface material. Accordingly, such surfaces can co-localize cells to be transduced (through the VLA-4 binding aptamer of the disclosure) and a viral vector (through the second DNA aptamer having binding affinity therefor) at the surface, thus improving the efficiency of transduction of the cell with the viral vector.

As used herein, “viral vectors” are viruses (e.g., lentivirus, retrovirus) that themselves include an polynucleotide and are capable of introducing that nucleotide sequence to a target cell through transduction. The person of ordinary skill in the art will appreciate that the surfaces of such viruses can include one or more molecules to which the second DNA aptamer can bind such as through non-covalent interactions (e.g., electrostatic interactions, hydrophobic interactions, shape complementation). In various embodiments as otherwise described herein, the second DNA aptamer has a binding affinity for the viral vector of at least 0.1 nM. For example, in certain such embodiments, the second aptamer has a binding affinity for the viral vector of at least 0.25 nM, or at least 0.5 nM, 0.75 nM, or at least 1 nM, or at least 2.5 nM, or at least 5 nM, or at least 10 nM, or at least 25 nM, or at least 50 nM, or at least 75 nM, or at least 100 nM. The method described in the Examples for determining binding to VLA-4 can be adapted by the person of ordinary skill in the art to determine binding affinity for the viral vector. The person of ordinary skill in the art can select a binding affinity that provides for a desirable degree of binding of a viral vector to the functionalized surface.

The person of ordinary skill in the art will appreciate that such aptamers (i.e., for both the viral vector) can be prepared according to generally known procedures such as, for example, a SELEX process. Processes analogous to that described in the Example below can be used. The person of ordinary skill in the art can select a binding affinity that provides for a desirable degree of binding of the viral vector to the inner surface of the container.

The second DNA aptamer can have a variety of lengths, depending on sequence and on the identity of the viral vector. In some embodiments, in a functionalized surface as otherwise described herein the second DNA aptamer has at least 10 nucleotides, e.g., at least 20 nucleotides, or at least 30 nucleotides. In some embodiments, in a functionalized surface as otherwise described herein the second DNA aptamer has no more than 200 nucleotides, e.g., no more than 150 nucleotides, or no more than 100 nucleotides. For example, in some embodiments, the second DNA aptamer has in the range of 10-200 nucleotides, e.g., 20-200, or 30-200, or 10-150, or 20-150, or 30-150, or 10-100, or 20-100, or 30-100 nucleotides.

The second DNA aptamer can be provided with a number of repeated sequences just as described above with respect to the VLA-4 binding DNA aptamer.

Notably, the DNA aptamer and the second DNA aptamer can be separately bound to the surface in separate polynucleotides, or, in other embodiments can be provided together in the same polynucleotide. This can be in an alternating series of repeats of the VLA-4 binding DNA aptamer and the second DNA aptamer, for example. Of course, in other embodiments, the polynucleotide comprises one or more blocks of two or more repeats of the VLA-4 binding aptamer in series, and one or more blocks of two or more repeats of the second DNA aptamer in series.

The second DNA aptamer can be bound to the surface (e.g., covalently or non-covalently) in manners analogous to those described above for the VLA-4 binding DNA aptamer.

Advantageously, the functionalized surfaces described herein can be provided as an inner surface of a container for cell transduction. Thus, another aspect of the disclosure is a container having, as an inner surface thereof, a functionalized surface as described herein. The containers of the disclosure can be provided in a number of forms. One especially convenient form is a cell culture bag, e.g., formed from one or more sheets of polymeric material as described herein. The person of ordinary skill in the art will be familiar with bag structures such as those used in cell culture, and will be able to adapt conventional bag structures for use in bags and methods of the disclosure based on the description herein Of course, the person of ordinary skill in the art will appreciate that the containers of the disclosure can be provided in a number of other forms, e.g., flasks, tubes, dishes. The person of ordinary skill in the art will appreciate that while in some embodiments substantially all inner surface area of the container can be provided as a functionalized surface as described herein, in other embodiments only a portion of inner surface area of the container is provided as a functionalized surface as described herein.

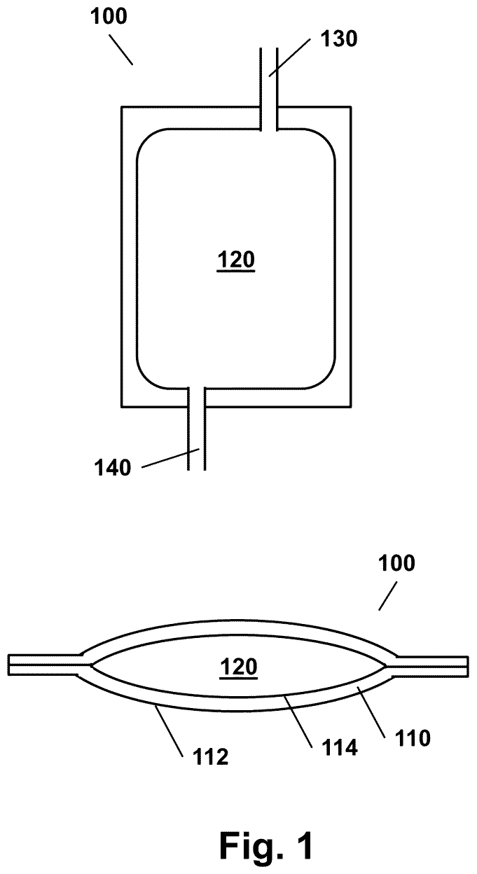

One embodiment of such a container, in the form of a cell culture bag, is shown in schematic top-down view (top) and cross-sectional view (bottom) in FIG. 1. Bag 100 of FIG. 1 includes a bag wall 110 having an outer surface 112 and an inner surface 114, with the inner surface 114 being provided as a functionalized surface of the disclosure. In some embodiments, both major inner surfaces of the bag is provided as functionalized surface; in other embodiments, only one major inner surface of the bag is provided as a functionalized surface. The bag 100 further includes ports 130 and 140, located at opposite ends of the bag for adding or removing media to or from the bag. The person of ordinary skill in the art will appreciate that the number and location of ports are not particularly limited, and accordingly can be positioned, for example, for convenience of use or manufacture. Bag 100 can be the product of bonding two fluoropolymer-containing sheets (e.g., two sheets having a layer of fluorinated ethylene propylene on an inside surface thereof) together at their edges (e.g., by laser welding, corona discharge, radiation, heat or melt lamination, etching, plasma treatment, wetting, adhesives, or combinations thereof) to form compartment 120. Ports 130 and 140 can be sealable to provide a sealed compartment 120.

Bag wall 110 can be uniform in its composition, or alternatively can include two or more distinct domains (e.g., two or more layers). For example, bonding two fluoropolymer sheets together, then coating the bonded sheets can provide an outer surface 112 differing in composition from inner surface 114. Similarly, bonding two multi-layer sheets together can provide an outer surface 112 differing in composition from inner surface 114. Multilayer sheets can be formed of both fluoropolymeric and nonfluorinated polymer materials; in such cases, a fluoropolymer layer can be provided at the inner surfaces of one or more of the multi-layer sheets. The thickness of bag wall 110, the volume of compartment 120, and the shape of bag 100 and/or compartment 120 are not particularly limited, and can be selected for convenience of use or manufacture, and/or to suit a specific application. For example, the thickness of the container wall can in various embodiments be within the range of 0.0003 inches to 0.2 inches, and the volume of the compartment can in various embodiments be within the range of 100 mL to 100 L.

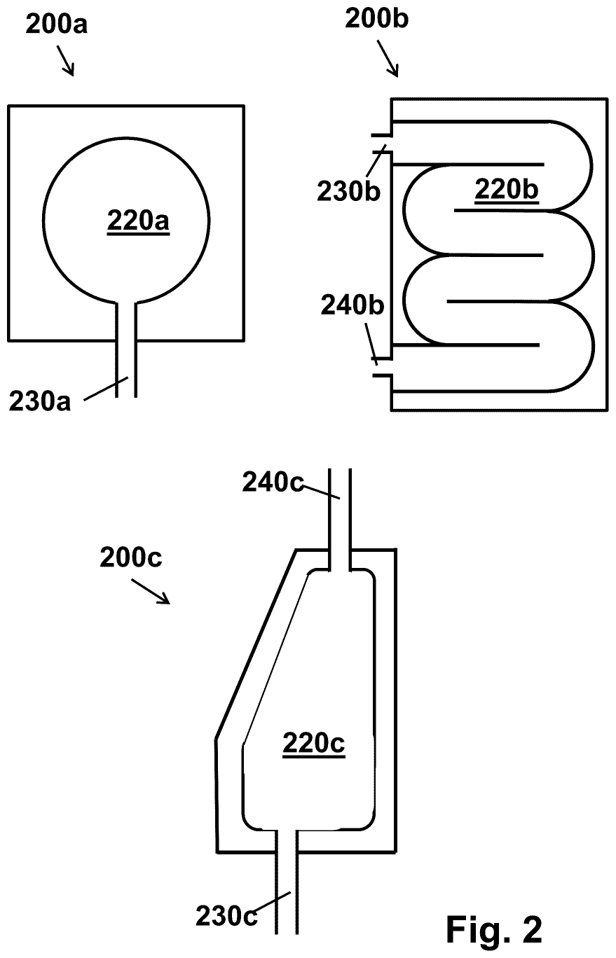

FIG. 2 shows several exemplary embodiments of configurations for culture bags suitable for use in the bags and methods of the disclosure. Bag 200a has only a single port 230a, providing access to compartment 220a. Bag 200b is in the so-called “serpentine” configuration, in which a longer path length through the system can be provided; ports 230b and 240b are connected by a serpentine path formed by serpentine-shaped compartment 220b formed by appropriate welding of the sheets forming the bag. And bag 200c has a non-rectangular shape, with a corresponding non-rectangular compartment 220c between ports 230c and 240c.

One or more of the walls of the container can be permeable to gases produced and consumed in a cell culture (e.g., O₂, CO₂) but impermeable to liquids (e.g., water). This can allow for passive exchange of gases across the container walls with the atmosphere to allow for respiration of cells in the bag.

The containers of the disclosure are desirably formed such that there is substantially no contamination of a fluid within the container. Accordingly, it is desirable for the inner surface of the container to be formed from materials that will not leach organics into the fluid. For example, in various embodiments as otherwise described herein, an inner surface of the container wall is formed of a polymer (e.g., a fluoropolymer such as fluorinated ethylene propylene) having a total organic carbon (TOC) in water of less than 0.1 mg/cm² (e.g., less than 0.05 mg/cm², or less than 0.05 mg/cm²). Such containers are described, e.g., in U.S. Patent Application Publications nos. 2016/0178490 and 2016/0178491, each of which is hereby incorporated herein by reference in its entirety; the person of ordinary skill in the art can, based on the description herein, adapt such containers for use in the containers and methods of the present disclosure.

As used herein, TOC is measured for a container employed in a system of the disclosure including, for example by extraction from an internal surface area of the container (with results reflected as mg/cm² are for the TOC per square centimeter of the internal area). TOC is measured according to US Pharmacopeia (USP) 643 and with equipment that utilizes a high temperature wet oxidation reaction of UV-promoted chemical oxidation (Ultra-Clean Technology Handbook: Volume 1: Ultra-Pure Water, Ohmi, Tadahiro; CRC Press, 1993, pp. 497-517). Purified water is placed in contact with the polymer for 24 hours at 70° C., for example at a ratio of 3 cm² of article surface area to 1 ml of water. The water is removed from contact with the polymer and tested in a TOC analyzer. A suitable piece of equipment is a TEKMAR DOHRMANN Model Phoenix 8000 TOC analyzer.

The containers described herein can advantageously be used for cell transduction using viral vectors. Accordingly, in various embodiments, the container has an aqueous medium disposed therein. The aqueous medium can be, for example, a cell culture medium. The person of ordinary skill in the art can select a desirable cell culture medium for a given cell type to be transduced.

The containers described herein can be used in the transduction and culture of cells. Accordingly, in various embodiments the container can have disposed therein, along with the aqueous medium, a population of cells having VLA-4 surface moieties. In various embodiments, the container can have disposed therein, along with the aqueous medium, one or more viral vectors (e.g., a lentivirus or a retrovirus), for example, together with the population of cells.

Advantageously, the present inventors have determined that containers described herein can facilitate co-localization of viral vectors and VLA-4-bearing target cells involved in a transduction process, desirably increasing the efficiency thereof, without requiring a user to perform a manual coating process shortly before conducting the transduction. Accordingly, another aspect of the disclosure is a method for transduction of a population of cells, comprising incubating one or more viral vectors and a population of cells having VLA-4 surface moieties in an aqueous medium (e.g., a cell culture medium) in a container as described herein. In certain such embodiments, the viral vector comprises a lentivirus or a retrovirus.

In various embodiments as otherwise described herein, the transduction method comprises including a suspension of the viral vector in a first aqueous medium (e.g., a viral vector supernatant) in the container; and then incubating the container comprising the viral vector for a first period of time. The first period of time can be any length sufficient to allow association of at least a portion of the viral vector with the second aptamer sequence of the inner surface of the container. For example, in various embodiments, the container comprising the viral vector is incubated for at least 1 hr., or at least 2 hr., or at least 3 hr., or at least 4 hr., or at least 5 hr., for example, at a temperature within the range of 32-37° C. After incubating for the first period of time, the method includes adding a population of cells having VLA-4 surface moieties (e.g., as a suspension in a second aqueous medium such as a cell culture medium) to the container; and then incubating the population of cells together with the viral vector in the container for a second period of for a second period of time. The second period of time can be any length sufficient to allow transduction of at least a portion of the population of cells. For example, in various embodiments, the viral vector and the population of cells are incubated together for at least 6 hr., or at least 12 hr., or at least 18 hr., or at least 1 day, or at least 1.5 days, or at least 2 days, or at least 2.5 days, for example, at a temperature within the range of 35-39° C. After incubation for the second period of time, the method includes collecting transduced cells from the container.

In certain such embodiments, the method further includes, after incubating the container for the first period of time, removing at least a portion of the first aqueous medium; then adding a wash medium to the container; and then removing at least a portion of the wash medium from the container (i.e., before adding the population of cells to the container).

In other embodiments, the transduction method comprises incubating a suspension of the viral vector and the population of cells having VLA-4 surface moieties in a first aqueous medium (e.g., a mixture of a viral vector supernatant and cell culture medium) in the container for a first period of time. The first period of time can be any length sufficient to allow transduction of at least a portion of the target cells. For example, in various embodiments, the container comprising the viral vector and the target cell is incubated for at least 6 hr., or at least 12 hr., or at least 18 hr., or at least 1 day, or at least 1.5 days, or at least 2 days, or at least 2.5 days, for example, at a temperature within the range of 35-39° C. After incubating for the first period of time, the method further includes collecting transduced cells from the container.

In various embodiments as otherwise described herein, collecting transduced cells includes removing a suspension of transduced cells from the container. In various embodiments, collecting transduced cells comprises adding a cell dissociation medium to the container (e.g., after removing at least a portion of the transduction medium from the container), and then removing a suspension of transduced cells in the cell dissociation medium from the container. The cell dissociation medium can include one or more dissociation agents capable of releasing target cells from the inner surface of the container. For example, in certain such embodiments, the cell dissociation media comprises one or more of salts and chelating agents (e.g., that can disrupt binding of the DNA aptamer to the VLA-4 surface moieties of the population of cells). In another example, the cell dissociation medium comprises one or more restriction enzymes (e.g., that can degrade the DNA aptamer). In another example, in certain such embodiments, the cell dissociation media comprises an oligo- or polynucleotide comprising a nucleotide sequence that is complimentary to the DNA aptamer (e.g., that can displace a bound cells from the DNA aptamer).

### Examples

1. Introduction

VLA-4 is a cell-surface integrin found on most leukocytes, but not neutrophils, that plays a role in cell adhesion of the movement of leukocytes through tissues. A DNA aptamer having high affinity and high specificity for binding of VLA-4 was developed using an aptamer enrichment strategy that produces high affinity and high specificity aptamers, using recombinant VLA-4 as the target and BSA as counter-target. The enriched library underwent sequencing and bioinformatics analysis to identify potential aptamers, which were synthesized on microarray for high-throughput assessment.

2. SELEX Strategy

2.1 Materials

Aptamer library, PCR primers, and biotinylated capture probe were all synthesized by Integrated DNA Technologies (Coralville, IA) and underwent desalting purification. MyOne T1 Dynabeads® (Life Technologies; Carlsbad, CA) streptavidin-coated magnetic beads were employed for partitioning steps. VLA-4 (designated P191; R&D Systems; Minneapolis, MN) was used as the target. Bovine Serum Albumin (BSA; Thermo Fisher Scientific; Waltham, MA) was used as counter-target. Jurkat cells (ATCC; Manassas, VA) were used as a representation of target in the context of live cells. 1×PBS, PH 7.4 was used as SELEX buffer.

2.2 Melting-Off SELEX

Aptamers were selected for target P191, with BSA used to reduce the enrichment of nonspecific aptamers. Melting-Off SELEX uses a complementary capture probe to immobilize a library to magnetic beads. Introduction of target, counter-target, or matrix can then be used to induce conformational changes in the library on binding, allowing for the partitioning of responsive sequences from non-responsive sequences.

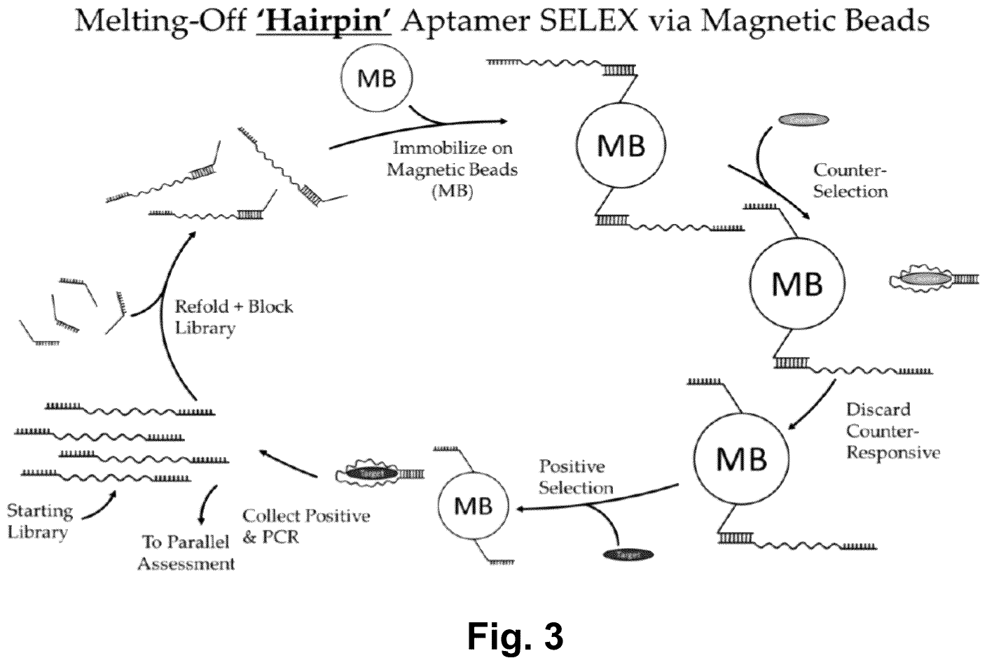

FIG. 3 provides an illustration of melting-off aptamer selection. Selection begins by refolding and blocking the library (left). After refolding is complete, the refolded library is captured on magnetic beads (top center). The beads can then be washed with 1×SELEX buffer, or incubated with counter-targets (counter-selection, top right). Species that change their conformation in response to counter-target are discarded (middle right). The beads are then washed with 1×SELEX buffer to ensure the sample is clear of residual counter-targets before beginning positive selection (bottom center). Species that change their conformation in response to the positive target are collected to undergo further experiments (bottom left).

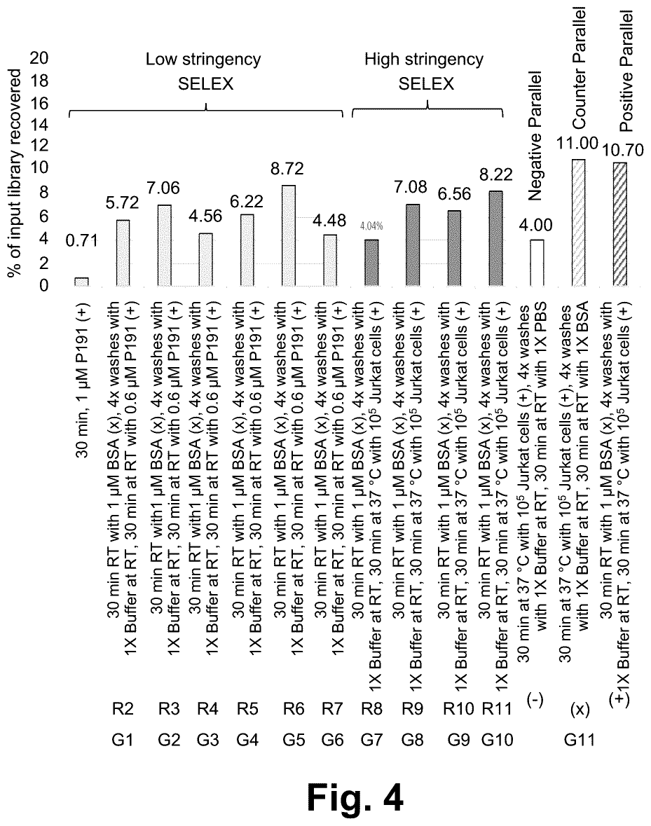

FIG. 4 provides a bar graph describing of melting-off based library enrichment towards P191 (+). Library recovery determined as a ratio between material recovered from the selection or parallel assessment step and the input amount of material. Specific selection conditions are indicated below bar graph.

A typical round of selection began with pre-incubating the library with the capture oligo at twice the library mole amount for refolding in 1×SELEX buffer by heating the sample to 90° C. for 1 minute, cooling to 60ºC for 5 minutes, and then cooling to 23° C. for 5 minutes. While the library was refolding, Dynabeads® were washed three times each with 1×PBS containing 0.01% Tween-20. After refolding was completed, the library was captured on the washed Dynabeads®. The Dynabeads® were then washed twice with 1×SELEX buffer to remove non-specifically bound library members. After the initial wash, the library was incubated with counter-targets (see FIG. 4 for details) at 37° C. Non-binding library was partitioned from counter-binding or buffer-responsive library by magnetic separation, after which the supernatant was discarded. The Dynabeads® were then washed repeatedly either washed again (number of washes described in FIG. 4) to remove any remaining non-specific library members. The remaining Dynabead®-bound-library was then incubated with positive target at 37° C. for 30 minutes. The recovered library was quantified before PCR amplification and propagation to the next round.

Library response was defined as the ratio of recovered material (determined by spectrophotometric analysis at A260) to the amount of input library. The initial round of selection omitted counter-target to maximize recovery of rare sequences from the starting library. Selection used 1 μM counter-target throughout. Target was used at 0.6 μM for the first seven rounds. When library response appeared to decrease, recombinant target was replaced by Jurkat cells to represent target as it would appear in the Client's final application. Once library response appeared to recover to that achieved in the low stringency selections, a parallel assessment was conducted. The responsive fraction of each sample was collected for sequencing and bioinformatics analysis. Detailed conditions of each selection round are provided in FIG. 4.

2.3 Parallel Assessment

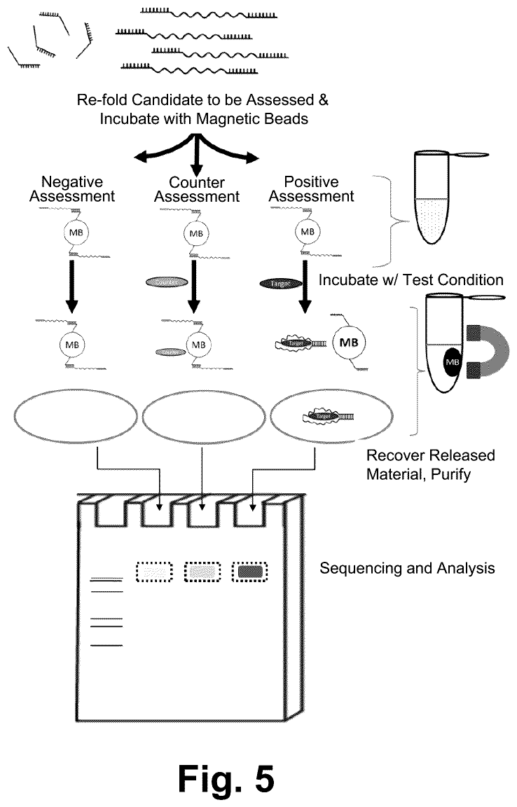

FIG. 5 provides an illustration of parallel assessment. The enriched library was divided into three equal portions for incubation with the following final conditions: buffer-only negative incubation (−), counter (x), and positive (+). Based on the strategy described in FIG. 3, condition-responsive species were released for collection during the final incubation. Bead-bound nonresponsive sequences will then be separated from responsive sequences by magnetic separation, and the supernatant containing responsive sequences was collected and prepared for sequencing. In an ideal scenario, only the positive samples will release responsive sequences while the counter-target and negative would have minimal material. In practice, a library that has significantly more library recovery in the positive samples over counter-target and negative is considered sufficiently enriched to identify successful aptamer sequences (see FIG. 4 for details).

The melting-off method was used to validate library enrichment (FIG. 5). The final enriched library pool is split into three equal portions: negative (−), counter (x), and positive (+). These portions undergo the same Melting-Off procedure but are exposed to their respective conditions in their final incubation. Responsive library material was recovered for sequencing and bioinformatics analysis.

3. Results

The results for 12 rounds of Melting-Off screening and parallel assessment are summarized in FIG. 4. The library recovery observed in Round 1 is typical, a result of collecting sequences that bind to the given target from over 1014 possible species. Library responded at expected levels from Rounds 2-6. When enrichment dropped in Round 7, target was changed from recombinant P191 to Jurkat cells to allow the library to recognize the target in the context of cells. The library recovered from this change by Round 11. At this point, the Round 12 (G11) library was taken to parallel assessment to collect material for sequencing and bioinformatics.

4. Phase IIa: Sequencing

The initial library was subjected to 12 rounds of Melting-Off selection and parallel assessment. The SELEX process is designed to enrich for sequences over multiple rounds of selection that bind to the given target of interest, in this case P191 protein, and remove sequences that respond to a general counter-target BSA. As a result, the population to be sequenced is expected to contain multiple copies of potential aptamer candidates.

The Illumina (San Diego, CA) MiniSeq system was implemented to sequence the aptamer libraries after the post-parallel selection using a single-end read technique. Deep sequencing and subsequent data analysis reduces the traditional approach of performing a large number of screening rounds, which may introduce error and bias due to the screening process (Schütze et al., 2011). Hundreds of thousands of sequences were analyzed from the parallel-exposed final libraries. From these sets of data, the library sequence families were constructed at 90% homology (sequence similarity considering mutations, deletions, and insertions).

| | | Sequence Families | |
|---|---|---|---|
| Target | Raw Sequences | (90% homology | Family Size |
| P191 | 719,767 | 500 | 101-7,565 |

5. Phase IIb: Bioinformatics and Aptamer Candidate Selection

Sequence family construction focused primarily on sequence similarity. This means that an individual sequence's frequency in the positive target population was factored in, but the degree of variation between similar sequences was also important, with 90% homology being the minimum requirement (100% match over the entire sequence is not necessary to join a family; up to 2 bases can be mismatched, inserted, or deleted). One would therefore expect families with the greatest number of members to rank highly as aptamer candidates. While this is true to an extent, there are a few other factors that adjust the importance of family size when determining which sequences are likely candidates.

The first factor is normally the presence of a sequence in the non-positive-target-exposed populations. Four libraries were collected for sequencing: the post-parallel assessment library that had been recovered from incubation with positive target in 1×SELEX Buffer (positive population G11(P); post-parallel assessment library recovered after incubation with counter-target in 1×SELEX Buffer (counter population G11(C)); post-parallel assessment library recovered after incubation with only 1×SELEX Buffer (negative population G11(N)); and the pre-parallel assessment library that had been recovered from incubation with positive target in 1×SELEX Buffer the round prior to the parallel assessment (pre-positive population G10(P)). The positive population was compared against the counter population to identify any sequences that were not removed during the counter selection steps, but still had some affinity for both the target and counter-target. Families were constructed from the positive population G11(P), then searched for primarily in the counter population. Those sequences that appeared at comparable rates in both positive and counter populations were ranked lower than ones that could predominantly be found in the positive populations. A similar process was used to compare candidates in the positive population (G11(P)) against the negative buffer-only population (G11(N)). These libraries showed little overlap between the final positive population and either the counter-target or negative populations. Instead, sequences were evaluated based on their frequency in G11(P) without being present in the counter-target population. Finally, the stability of a candidate's secondary structure was used as a “tiebreaker” parameter. As such, while secondary structure is not a primary factor, they influence which of several candidates with similar occurrence rates are selected for further analysis. After considering these factors, 200 candidates were chosen for further assessment.

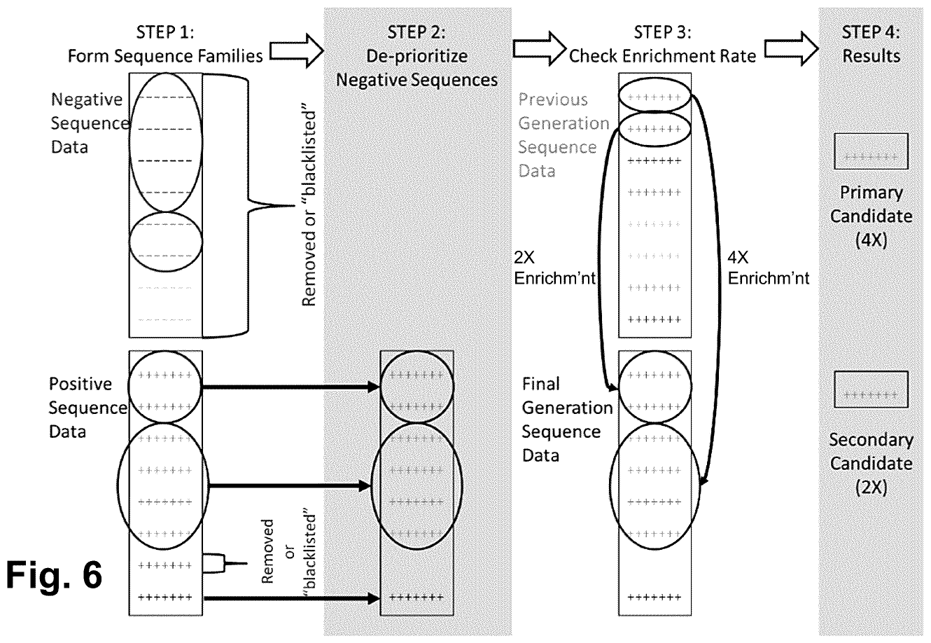

FIG. 6 describes bioinformatics & candidate selection. (Step 1) Sequence data from the ultimate (i.e. post-parallel) round of selection are constructed into families with >90% homology using the FASTAptamer algorithm. Characters with the same color in this figure represent members of the same sequence family. (Step 2) Families that are common to both the negative and positive final generation data sets are removed from the candidate pool. (Step 3-4) Remaining sequence families are compared against the penultimate (i.e. parallel) generation families to determine enrichment rate—families with the highest enrichment rates are the more highly-ranked candidates for further analysis.

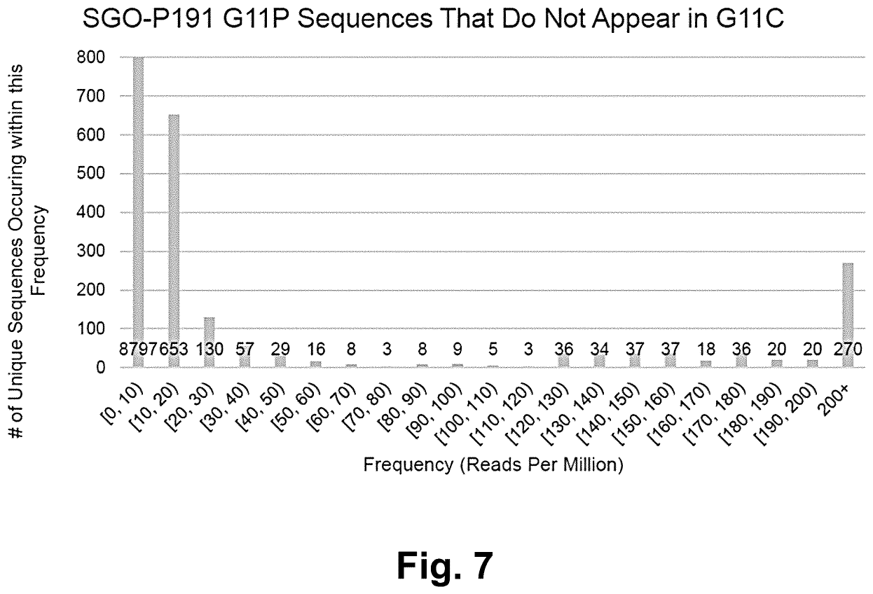

FIG. 7 is a graph of distribution of family frequencies from the P191(+) G11(P) library that do not Appear in G11(C). Plot of the frequency of sequences discovered in the final generation positive target-(G11(P)) that do not appear in the counter-target-(G11(C)) exposed libraries. G11(P) normalized sequence frequencies are on the x-axis; number of unique sequences with the given frequencies are on the y-axis.

6. Phase III: Microarray Synthesis and Semi-Quantitative Assessment

6.1 Microarray Methods

A Cy5-labeled reporter oligo complimentary to a constant region of the library (5′-GTC GTC CCG AGA GCC TCG/3Cy5Sp/-3′) was synthesized by IDT (Coralville, IA) and purified by desalting. This oligo would be displaced during target binding to provide an indication of binding ability.

The selected full-length aptamer candidates were arranged randomly by name before being synthesized by LC Sciences (Houston, TX) on duplicate 4K microarray chips using standard phosphoramidite chemistry and masking techniques proprietary to LC Sciences. Each candidate cluster occupied 18 positions (3×6 colonies), with control sequences distributed semi-regularly throughout each chip. Monoclonal colonies of each candidate were synthesized 3′ to 5′, with the 3′-end anchoring each individual candidate to the colony region on the chip. Colonies are defined as aptamer replicates in a single well, while clusters are defined as the group of wells that contain the same candidate. Candidate colonies and clusters are needed to significantly enhance the change in signal when responding to target or counter target, as an individual molecule's change in fluorescence can be drowned out by the signal of its neighbors. Additional sequences were synthesized as a quality control measure.

Before use, the microarray chip was blocked with 0.1% BSA in 1×PBS solution to prevent nonspecific reporter binding to the chip. Reporter sequence was annealed to candidates using a modified protocol, as the microarray setup cannot be heated above 60° C. without compromising the microfluidic chip. A solution of 100 nM Cy5-labeled Reporter oligo in 1×SELEX Buffer without BSA was introduced to the chip with an oscillating peristaltic pump to ensure even distribution across colonies. During this process, the candidates were denatured by heating the chip and solution to 60ºC for 20 minutes and then cooling to 23° C. for 20 minutes, allowing the reporter to anneal. Fluorescence readings were taken by a GenePix 4000B Microarray Scanner (Molecular Devices, LLC.; San Jose, CA) to establish baseline responses of each candidate. Once baseline images were taken, 400 μL of either 1 μM positive target P191(+) in 1×SELEX Buffer or 1 μM counter-target BSA(X) in 1×SELEX Buffer was then circulated inside the chip for 16 hours at 37° C. Post-incubation, the chip was flushed twice with 500 μL of 1×SELEX Buffer. 1×SELEX Buffer from the second wash was left in place during pre-incubation and post-incubation fluorescence scanning.

Data analysis was conducted as follows. The mean background fluorescence value (fbackground) was subtracted from the mean fluorescence value of each candidate prior to the addition of sample (fpre-treatment) as well as each candidate after the addition of sample (fpost-treatment). The amount of signal loss from the pre-treatment condition to the post-treatment condition was then expressed as a percentage. This process was repeated for the target and counter-target assessment. The percent signal loss for the target assessment was then divided by the percent signal loss for the counter assessment to generate a score. A score greater than one indicates increased response to the target condition over the counter condition. The percent signal loss from the target assessment minus the percent signal loss from the counter assessment is also displayed.

$$[ 1 - \frac{( {f}_{post - treatment} - {f}_{background} )}{( {f}_{pre - treatment} - {f}_{background} )} ] * 100 = \% Fluorescence Decrease \qquad Equation 1$$

$$\frac{\% Fluorescence {Decrease}_{target}}{\% Fluorescence {Decrease}_{counter}} = Score \qquad Equation 2$$

6.2 Microarray Results

Candidates were first tested against 1 μM positive target in 1×SELEX Buffer. Some candidates interacted with the Reporter better than others. After this reading was taken, candidates were incubated with target for 16 hours at 37° C. The solution at the inlet of the peristaltic pump was then replaced with 1×SELEX Buffer to wash the target sample from the microarray.

Fluorescence readings of each pixel were collected and averaged to produce a Mean Fluorescence value and Standard Deviation per candidate (data available in separate file). Candidate response to target was then calculated as described in Equation 1. The same processes were used to analyze candidate response to 1 μM counter-target in 1×SELEX Buffer.

Candidate percent responses to target sample and counter target sample were calculated as the mean of the 18 replicate positions pre- and post-incubation, omitting colonies that did not successfully incorporate the fluorescent probe. Pre-incubation fluorescence was then divided by post-incubation fluorescence to produce a Signal Ratio value, which was used to rank how responsive a sequence was to the assessment condition; more aptamer response would result in greater displacement of the fluorescent Reporter, and therefore a larger difference in pre- and post-incubation readings. Signal Ratio values of around 2 are typical, while values of <1.5 are of concern. The top 5 candidates from the target-assessed microarrays have Signal Ratio values of at least 1.7 (data available in separate file) which is promising. The Signal Ratio values of the top 5 responsive sequences to counter-targets are >50 (data available in separate file) —unusually high and worthy of further examination.

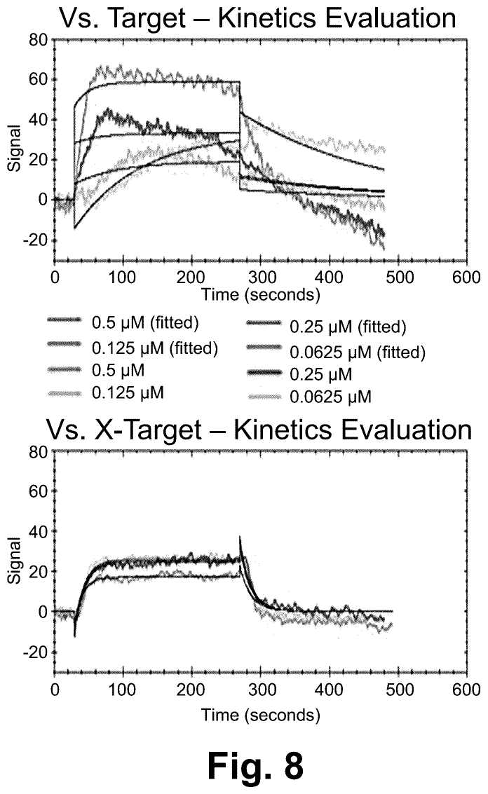

The percent responses themselves were then compared to determine candidates that specifically responded to the target. Candidates were ranked according to the ratio of signal loss to the target condition against the signal loss to the counter condition. The greater this score, the more response to the target condition relative to the response to the counter condition. As before, scores >2 are favorable, while scores <1.5 are of concern—the top 5 under this criterion had scores >25. Aptamer candidates identified for P191(+) target (FIG. 8) were then synthesized for Phase IV qualitative assessment before characterization by Surface Plasmon Resonance (SPR).

A collection of aptamer candidates against P191(+). A-E was thus developed. Notably, the general structure for many of these included PCR primer annealing regions at both the 5′ end and at the 3′ end, with a unique sequence in between (5′-CGA GGC TCT CGG GAC GAC-[sequence]-GTC GTC CCG CCT TTA GGA TTT ACA G-3′).

7. Phase V: Qualitative Validation

7.1 Assessment Method

Various candidates were synthesized with 5′-biotin for attachment to SPR sensor chips and purified by desalting. Assessment followed a different method than that used in Phase I SELEX. Briefly, candidates were individually prepared by suspending 200 pmoles of candidate or G0 naïve DNA library (as a control) in 1×SELEX Buffer in total volume of 200 μL. Samples were mixed well, then refolded as described in Phase I before injecting to be captured on SPR sensor chips. Once a stable baseline was achieved post-loading, increasing concentrations (0.03125 μM, 0.0625 μM, 0.125 μM, 0.25 μM, and 0.5 μM) of either target P191(+) or counter-target BSA were injected in series at room temperature to measure aptamer-target association, followed by switching to buffer only to measure dissociation. Results from the target and counter-target against control library were then subtracted from the corresponding candidate runs prior to kinetics analysis and binding affinity fitting (FIG. 9). Specificity was then calculated as the ratio of counter-targeting binding affinity to target binding affinity.

7.2 Assessment Results

In addition to running each candidate against the full concentration series of counter-target and target, an unselected random library (negative control) was also assessed. The unselected library responses were taken as a background signal to be subtracted from candidate response curves.

FIG. 8 provides graphs of SPR-based assessment of SGO-P06701 against target and counter-target. (Top) Injections of varying concentrations of target P191(+) against candidate SGO-P06701 immobilized on SPR sensor surface. (Bottom) Candidate responses to counter-target at various concentrations.

Most candidates did not respond to counter-target. The responses to target are more promising, with SGO-P06701 (i.e., having SEQ ID NO: 1) producing raw signal changes more than twice that of the control DNA. After controlling for background and fitting to a 1:1 binding model, SGO-P06701 was found to have a binding affinity of 59.2 nM to VLA-4, and an affinity of 1.74 μM to BSA). Based on these values, its specificity for the target was 29.4×.

8. Conclusions

A SELEX experiment provided a library of sequences. Once the library responded to target in the context of cells, parallel assessment was conducted to collect material for sequencing. The top 200 sequences identified after sequencing and bioinformatics were semi-quantitatively assessed for binding, at which point the top 5 candidates were chosen for synthesis at analytical scale with 5′-biotin for immobilization on SPR sensor chips. Candidates and control DNA were separately assessed against concentrations of target P191(+) and counter-target BSA to determine association and dissociation rates, and therefore binding affinity constants. While most candidates did not respond to counter-target, they also displayed weak response to target. Candidate SGO-06701 appears to be the most promising, with a Kd value of 59.2 nM against target VLA-4 and a Kd of 1.74 μM against counter-target BSA (binding specificity of 29.4× for target).

Additional aspects of the disclosure are provided by the following enumerated embodiments, which can be combined in any number and in any fashion that are not technically or logically incompatible.

Embodiment 1. A DNA aptamer comprising a plurality of nucleotides, the DNA aptamer having at least 80% sequence identity with the sequence of SEQ ID NO: 1 (AAACTGCAGCGATTCATTAGTACGGCCTTT).
Embodiment 2. The DNA aptamer of embodiment 1, having at least 86.6% sequence identity with the sequence of SEQ ID NO: 1.
Embodiment 3. The DNA aptamer of embodiment 1, having at least 90% sequence identity with the sequence of SEQ ID NO: 1.
Embodiment 4. The DNA aptamer of embodiment 1, having at least 93.3% sequence identity with the sequence of SEQ ID NO: 1.
Embodiment 5. The DNA aptamer of embodiment 1, having at least 96.6% sequence identity with the sequence of SEQ ID NO: 1.
Embodiment 6. The DNA aptamer of embodiment 1, having 100% sequence identity with the sequence of SEQ ID NO: 1.
Embodiment 7. The DNA aptamer of any of embodiments 1-6, wherein additional nucleotides not found in the sequence of SEQ ID NO: 1 are present.
Embodiment 8. The DNA aptamer of any of embodiments 1-6, wherein additional nucleotides not found in the sequence of SEQ ID NO: 1 are not present.
Embodiment 9. The DNA aptamer of any of embodiments 1-8, having at least one span of 10 contiguous nucleotides in common with the sequence of SEQ ID NO: 1, e.g., one such span, or two such spans, or three such spans.
Embodiment 10. The DNA aptamer of any of embodiments 1-8, having at least one span of 15 contiguous nucleotides in common with the sequence of SEQ ID NO: 1, e.g., one such span, or two such spans.
Embodiment 11. The DNA aptamer of any of embodiments 1-8, having a span of 20 contiguous nucleotides in common with the sequence of SEQ ID NO: 1.
Embodiment 12. The DNA aptamer of any of embodiments 1-8, having a span of 26 contiguous nucleotides in common with the sequence of SEQ ID NO: 1.
Embodiment 13. The DNA aptamer of any of embodiments 1-8, having a span of 50 contiguous nucleotides in common with the sequence of SEQ ID NO: 1.
Embodiment 14. The DNA aptamer of any of embodiments 1-8, having a span of 60 contiguous nucleotides in common with the sequence of SEQ ID NO: 1.
Embodiment 15. The DNA aptamer of any of embodiments 1-8, having a span of 70 contiguous nucleotides in common with the sequence of SEQ ID NO: 1.
Embodiment 16. The DNA aptamer of any of embodiments 1-15, having at least 24 nucleotides, e.g., at least 26 nucleotides, or at least 27 nucleotides, or at least 28 nucleotides, or at least 29 nucleotides, or at least 30 nucleotides.
Embodiment 17. The DNA aptamer of any of embodiments 1-16, having no more than 200 nucleotides, e.g., no more than 150 nucleotides, or no more than 100 nucleotides, or no more than 80 nucleotides, or no more than 75 nucleotides, or no more than 74 nucleotides, or no more than 73 nucleotides.
Embodiment 18. The DNA aptamer of any of embodiments 1-15, having in the range of 24-200 nucleotides, e.g., 26-200, or 27-200, or 28-200, or 29-200, or 30-200, or 24-150, or 26-150, or 27-100, or 28-100, or 29-100, or 30-100, or 24-75, or 26-75, or 27-75, or 28-75, or 29-75, or 30-75, or 24-50, or 26-50, or 27-50, or 28-50, or 29-50, or 30-50, or 24-40, or 26-40, or 27-40, or 28-40, or 29-40, or 30-40 nucleotides.
Embodiment 19. The DNA aptamer of any of embodiments 1-15, having in the range of 24-36 nucleotides, e.g., 26-36, or 28-36, or 30-36, or 24-34, or 26-34, or 28-34, or 30-34, or 24-32, or 26-32, or 28-32, or 30-32, or 24-30, or 26-30, or 28-30, or 30 nucleotides.
Embodiment 20. A DNA aptamer comprising a plurality of nucleotides, the DNA aptamer having at least 80% sequence identity with the sequence of SEQ ID NO: 2 (CGAGGCTCTCGGGACGACAAACTGCAGCGATTCATTAGTACGGCCTTTGTCGTCCCG CCTTTAGGATTTACAG).
Embodiment 21. The DNA aptamer of embodiment 20, having at least 85% sequence identity with the sequence of SEQ ID NO: 2.
Embodiment 22. The DNA aptamer of embodiment 20, having at least 90% sequence identity with the sequence of SEQ ID NO: 2.
Embodiment 23. The DNA aptamer of embodiment 20, having at least 91.7% (e.g., at least 93.1%, or at least 94.5%) sequence identity with the sequence of SEQ ID NO: 2.
Embodiment 24. The DNA aptamer of embodiment 20, having at least 95.8% (e.g., at least 97.2%, or at least 98.6%) sequence identity with the sequence of SEQ ID NO: 2.
Embodiment 25. The DNA aptamer of embodiment 20, having 100% sequence identity with the sequence of SEQ ID NO: 2.
Embodiment 26. The DNA aptamer of any of embodiments 20-25, wherein additional nucleotides not found in the sequence of SEQ ID NO: 2 are present.
Embodiment 27. The DNA aptamer of any of embodiments 1-6, wherein additional nucleotides not found in the sequence of SEQ ID NO: 2 are not present.
Embodiment 28. The DNA aptamer of any of embodiments 20-27, having at least one span of 10 contiguous nucleotides in common with the sequence of SEQ ID NO: 1, e.g., one such span, or two such spans, or three such spans, or four such spans, or five such spans, or six such spans.
Embodiment 29. The DNA aptamer of any of embodiments 20-27, having at least one span of 20 contiguous nucleotides in common with the sequence of SEQ ID NO: 1, e.g., one such span, or two such spans, or three such spans.
Embodiment 30. The DNA aptamer of any of embodiments 20-27, having at least one span of 30 contiguous nucleotides in common with the sequence of SEQ ID NO: 1, e.g., one such span, or two such spans.
Embodiment 31. The DNA aptamer of any of embodiments 20-27, having a span of 40 contiguous nucleotides in common with the sequence of SEQ ID NO: 1.
Embodiment 32. The DNA aptamer of any of embodiments 20-27, having a span of 50 contiguous nucleotides in common with the sequence of SEQ ID NO: 1.
Embodiment 33. The DNA aptamer of any of embodiments 20-27, having a span of 60 contiguous nucleotides in common with the sequence of SEQ ID NO: 1.
Embodiment 34. The DNA aptamer of any of embodiments 20-27, having a span of 70 contiguous nucleotides in common with the sequence of SEQ ID NO: 1.
Embodiment 35. The DNA aptamer of any of embodiments 20-34, having at least 60 nucleotides, e.g., at least 65 nucleotides, or at least 70 nucleotides, or at least 71 nucleotides, or at least 72 nucleotides, or at least 73 nucleotides.
Embodiment 36. The DNA aptamer of any of embodiments 20-34, having no more than 200 nucleotides, e.g., no more than 150 nucleotides, or no more than 100 nucleotides, or no more than 80 nucleotides, or no more than 75 nucleotides, or no more than 74 nucleotides, or no more than 73 nucleotides.
Embodiment 37. The DNA aptamer of any of embodiments 20-34, having in the range of 60-200 nucleotides, e.g., 65-200, or 70-200, or 71-200, or 72-200, or 73-200, or 60-150, or 70-150, or 71-150, or 72-150, or 73-150, or 60-100, or 70-100, or 71-100, or 72-100, or 73-100, or 60-80, or 70-80, or 71-80, or 72-80, or 73-80, or 60-75, or 65-75, or 70-75, or 60-74, or 65-74, or 70-74, or 60-73, or 65-73 or 70-73 nucleotides.
Embodiment 38. The DNA aptamer of any of embodiments 20-34, having in the range of 71-75 nucleotides, e.g., 72-75, or 73-75, or 71-74, or 72-74, or 73-74, or 71-73, or 72-73, or 73 nucleotides.
Embodiment 37. The DNA aptamer of any of embodiments 1-36, wherein the DNA aptamer has a binding affinity of at least 0.1 nM for VLA-4, e.g., at least 0.25 nM, or at least 0.5 nM, 0.75 nM, or at least 1 nM, or at least 2.5 nM, or at least 5 nM, or at least 10 nM, or at least 25 nM, or at least 50 nM, or at least 75 nM, or at least 100 nM.
Embodiment 38. The DNA aptamer of any of embodiments 1-37, further comprising a reactive group-terminated linker bound to the plurality of nucleotides.
Embodiment 39. The DNA aptamer of any of embodiment 38, wherein the reactive group is a primary amine.
Embodiment 40. The DNA aptamer of embodiment 38, wherein the reactive group is a carboxylate.
Embodiment 41. The DNA aptamer of any of embodiments 38-40, wherein the linker is a difunctional hydrocarbon, or a difunctional polyether.
Embodiment 42. The DNA aptamer of any of embodiments 38-41, wherein the linker is covalently bound to the nucleotides via a phosphodiester bond.
Embodiment 43. The DNA aptamer of any of embodiments 38-42, wherein the linker separates the reactive group from an atom of the nucleotides of the aptamer by no more than 200 bonds, e.g., no more than 150 bonds, or no more than 100 bonds, or no more than 50 bonds, or no more than 25 bonds.
Embodiment 44. The DNA aptamer of any of embodiments 38-43, wherein the linker separates the reactive group from an atom of the nucleotides of the aptamer by at least 4 bonds, e.g., at least 6 bonds, or at least 8 bonds, or at least 10 bonds.
Embodiment 45. The DNA aptamer of any of embodiments 38-44, wherein the linker separates the reactive group from an atom of the nucleotides of the aptamer by in the range of 4-200 bonds, e.g., 6-200, or 8-200, or 10-200, or 4-150, or 6-150, or 8-150, or 10-150, or 4-100, or 6-100, or 8-100, or 10-100, or 4-50, or 6-50, or 8-50, or 10-50, or 4-25, or 6-25, or 8-25, or 10-25 bonds.
Embodiment 46. A functionalized surface comprising
a surface material; and
bound to the surface material, a DNA aptamer of any of embodiments 1-45.
   Embodiment 47. The functionalized surface of embodiment 46, wherein the surface material is a fluoropolymer.
   Embodiment 48. The functionalized surface of embodiment 47, wherein the fluoropolymer is polytetrafluoroethylene (PTFE), perfluoroalkoxy (PFA), ethylene tetrafluoroethylene (ETFE), polyvinylidene fluoride (PVDF), polychlorotrifluoroethylene (PCTFE), ethylene chlorotrifluoroethylene (ECTFE), fluorinated ethylene propylene (FEP), ethylene fluorinated ethylene propylene (EFEP), perfluoropolyether (PFPE), modified polytetrafluoroethylene (TFM), polyvinyl fluoride (PVF), or any mixture thereof.
   Embodiment 49. The functionalized surface of embodiment 47, wherein the fluoropolymer is a fluorinated ethylene propylene polymer.
   Embodiment 50. The functionalized surface of any of embodiments 46-49, wherein the DNA aptamer is covalently bound to the surface material.
   Embodiment 51. The functionalized surface of embodiment 50, wherein the DNA aptamer is covalently bound to the surface material through reaction of a reactive group with a functional group of the surface material.
   Embodiment 52. The functionalized surface of embodiment 51, wherein the reaction of the reactive group with the functional group of the surface material forms a phosphodiester, an amide, or an amine covalently bonding the DNA aptamer to the surface material (e.g., through a linking group).
   Embodiment 53. The functionalized surface of any of embodiments 46-52, further comprising a second DNA aptamer bound to the surface material, the second DNA aptamer having a binding affinity for a viral vector.
   Embodiment 54. The functionalized surface of embodiment 53, wherein the second DNA aptamer has a binding affinity of at least 0.1 nM for the viral vector, e.g., at least 0.25 nM, or at least 0.5 nM, 0.75 nM, or at least 1 nM, or at least 2.5 nM, or at least 5 nM, or at least 10 nM, or at least 25 nM, or at least 50 nM, or at least 75 nM, or at least 100 nM.
   Embodiment 55. The functionalized surface of embodiment 53 or embodiment 54, wherein the second DNA aptamer has at least 10 nucleotides, e.g., at least 20 nucleotides, or at least 30 nucleotides.
   Embodiment 56. The functionalized surface of any of embodiments 53-55, wherein the second DNA aptamer has no more than 200 nucleotides, e.g., no more than 150 nucleotides, or no more than 100 nucleotides.
   Embodiment 57. The functionalized surface of embodiment 53 or embodiment 54, wherein the second DNA aptamer has in the range of 10-200 nucleotides, e.g., 20-200, or 30-200, or 10-150, or 20-150, or 30-150, or 10-100, or 20-100, or 30-100 nucleotides.
   Embodiment 58. The functionalized surface of any of embodiments 53-57, wherein the viral vector is a lentivirus or a retrovirus.
   Embodiment 59. A container comprising, as an inner surface thereof, a functionalized surface according to any of embodiments 46-58.
   Embodiment 60. A container of embodiment 59, in the form of a cell culture bag.
   Embodiment 61. A container of embodiment 59 or embodiment 60, having disposed therein an aqueous medium such as a cell culture medium.
   Embodiment 62. A container of embodiment 61, having disposed therein a population of cells having VLA-4 surface moieties.
   Embodiment 63. A container of embodiment 61 or embodiment 62, having disposed therein one or more viral vectors (e.g., a lentivirus or a retrovirus).
   Embodiment 64. A method for transduction of a population of cells, the method comprising incubating one or more viral vectors and a population of cells having VLA-4 surface moieties in an aqueous medium (e.g., a cell culture medium) in a container according to embodiment 59 or embodiment 60.
   Embodiment 65. The method of claim 64, wherein the one or more viral vectors is a lentivirus or a retrovirus).
   Embodiment 66. The method of embodiment 64 or embodiment 65 comprising
providing a suspension of the viral vector in a first aqueous medium to the container;
incubating the container comprising the viral vector for a first period of time;
adding the population of cells (e.g., as a suspension in a second aqueous medium) to the container;
incubating the population of cells and the one or more viral vectors for a second period of time; and then
collecting transduced cells from the container.
   Embodiment 67. The method of embodiment 66, further comprising, after incubating the container for the first period of time
removing at least a portion of the first aqueous medium; then
adding a wash medium to the container; then
removing at least a portion of the wash medium from the container; and then
adding the population of cells to the container.
   Embodiment 68. The method of embodiment 66 or embodiment 67, comprising
providing a suspension of the one or more viral vectors and the population of cells in a first aqueous medium to the container;
incubating the one or more viral vectors and the population of cells in the container for a first period of time; and then
collecting transduced cells from the container.
   Embodiment 69. The method of any of embodiments 66-68, wherein collecting transduced cells from the container comprises
adding a cell dissociation medium to the container; and then
removing a suspension of transduced cells from the container.
   Embodiment 70. The method of embodiment 69, wherein the cell dissociation medium comprises one or more of salts and chelating agents.
   Embodiment 71. The method of embodiment 69, wherein the cell dissociation medium comprises one or more restriction enzymes.
   Embodiment 72. The method of embodiment 69, wherein the cell dissociation medium comprises an oligo- or polynucleotide having a nucleotide sequence that is complementary to the DNA aptamer.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice. Thus, before the disclosed processes and devices are described, it is to be understood that the aspects described herein are not limited to specific embodiments, apparatuses, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. It will be further understood that the endpoints of each of the ranges are significant both in relation to the other endpoint, and independently of the other endpoint.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Numerous references have been made to patents and printed publications throughout this specification. Each of the cited references and printed publications are individually incorporated herein by reference in their entirety.

Furthermore, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A DNA aptamer comprising a plurality of nucleotides, the DNA aptamer having at least 80% sequence identity with the sequence of SEQ ID NO: 1 (AAACTGCAGCGATTCATTAGTACGGCCTTT).

2. The DNA aptamer of claim 1, having at least 93.3% sequence identity with the sequence of SEQ ID NO: 1.

3. The DNA aptamer of claim 1, having a span of 20 contiguous nucleotides in common with the sequence of SEQ ID NO: 1.

4. A DNA aptamer comprising a plurality of nucleotides, the DNA aptamer having at least 80% sequence identity with the sequence of SEQ ID NO: 2 (CGAGGCTCTCGGGACGACAAACTGCAGCGATTCATTAGTACGGCCTTTGTCGTCCCG CCTTTAGGATTTACAG).

5. The DNA aptamer of claim 4, having at least 91.7% (e.g., at least 93.1%, or at least 94.5%) sequence identity with the sequence of SEQ ID NO: 2.

6. The DNA aptamer of claim 4, having at least one span of 30 contiguous nucleotides in common with the sequence of SEQ ID NO: 1.

7. The DNA aptamer of claim 1, further comprising a reactive group-terminated linker bound to the plurality of nucleotides.

8. The DNA aptamer of claim 7, wherein the reactive group is a primary amine or a carboxylate.

9. A functionalized surface comprising
a surface material; and
bound to the surface material, a DNA aptamer of claim 1.

10. The functionalized surface of claim 9, wherein the surface material is a fluoropolymer.

11. The functionalized surface of claim 9, wherein the DNA aptamer is covalently bound to the surface through reaction of a reactive group with a functional group of the surface material.

12. The functionalized surface of claim 11, wherein the reaction of the reactive group with the functional group of the surface material forms a phosphodiester, an amide, or an amine covalently bonding the DNA aptamer to the surface material.

13. The functionalized surface of claim 9, further comprising a second DNA aptamer bound to the surface material, the second DNA aptamer having a binding affinity for a viral vector, the second DNA aptamer having a binding affinity of at least 10 nM for the viral vector.

14. The functionalized surface of claim 13, wherein the viral vector is a lentivirus or a retrovirus.

15. A container comprising, as an inner surface thereof, a functionalized surface according to claim 9.

16. A container of claim 15, in the form of a cell culture bag.

17. A container of claim 59 or claim 60, having disposed therein a cell culture medium and a population of cells having VLA-4 surface moieties.

18. A method for transduction of a population of cells, the method comprising incubating one or more viral vectors and a population of cells having VLA-4 surface moieties in an aqueous medium in a container according to claim 17.

19. The method of claim 18, wherein the one or more viral vectors is a lentivirus or a retrovirus).


SEQUENCE LISTING (2 sequences)

SEQ ID NO: 1 - DNA, 30 bp, synthetic construct
  source 1..30: mol_type = other DNA
aaactgcagcgattcattagtacggccttt

SEQ ID NO: 2 - DNA, 73 bp, synthetic construct
  source 1..73: mol_type = other DNA
cgaggctctcgggacgacaaactgcagcgattcattagtacggcctttgtcgtcccgcctttaggatttacag